(19) United States
(12) Patent Application Publication
(10) Pub. No.: US 2026/0351028 A1
(43) Pub. Date: Nov. 5, 2026

(54) FULLY AUTOMATED DIGITAL FINGERPRINTING OF CELLULAR STRUCTURES

(71) Applicants: THE BRIGHAM AND WOMEN'S HOSPITAL, INC., Boston, MA (US); THE GENERAL HOSPITAL CORPORATION, Boston, MA (US)

(72) Inventors: Hadi Shafiee, Boston, OH (US); Prudhvi Thirumalaraju, Boston, MA (US); Charles L. Bormann, Boston, MA (US); Manoj K. Kanakasabapathy, Boston, MA (US); Sai Hemanth Kumar Kandula, Boston, MA (US)

(21) Appl. No.: 19/487,102

(22) PCT Filed: May 31, 2024
(86) PCT No.: PCT/US2024/032002

§ 371 Date: Nov. 24, 2025

Related U.S. Application Data
(60) Provisional application No. 63/505,123, filed on May 31, 2023.

(51) Int. Cl. G06V 20/69; G06F 40/166; G06V 10/26; G06V 10/74; G06V 10/764; G06V 10/82; G06V 20/40; G16H 15/00

(52) U.S. Cl. CPC G06V 20/698; G06V 10/26; G06V 10/761; G06V 10/764; G06V 10/82; G06V 20/46; G06V 20/695; G16H 15/00; G06F 40/166; G06V 2201/03

(57) ABSTRACT

Systems and methods are provided for digital witnessing of cellular structures. A system includes a processor and a non-transitory computer readable medium that stores instructions executable by the processor to provide an image interface configured to receive a first image and a similarity network. The similarity network compares the first image to a second image to determine a probability that the first and second images represent a same cellular structure or line of cellular structures. The similarity network is trained on a training set of samples, each including two images and a binary label. The training set includes positive examples, using two images of a same cellular structure or line of cellular structures, and negative examples, which use two images that are not of the same structure or line of cellular structures. An output device receives the probability or a categorical parameter representing the probability from the similarity network.

### RELATED APPLICATION

The present application claims priority to U.S. Provisional Patent Application Ser. No. 63/505,123 filed May 31, 2023, and entitled METHOD AND SYSTEM FOR FINGERPRINTING AND TRACKING CELLS VIA IMAGE SIMILARITY METRICS AND MACHINE LEARNING. The entire content of this application is incorporated herein by reference in its entirety for all purposes.

### STATEMENT ON GOVERNMENT RIGHTS

This invention was made with government support under award numbers R01AI138800-05, 1R01EB033866-01 and 4R33AI140489-04 from the National Institutes of Health and National Institute of Biomedical Imaging and Bioengineering. The government has certain rights in the invention.

### TECHNICAL FIELD

This invention relates to medical imaging systems, and more particularly, to fully automated digital fingerprinting of cellular structure.

### BACKGROUND

In vitro fertilization (IVF), by its nature, will always remain medically and emotionally complex and medical errors can be tragic for patients and their families. Each year over 2.5 million IVF cycles are performed worldwide, and each cycle involves several procedures including the procurement, manipulation, evaluation and culture of gametes and embryos, all with different risk profiles and potential failures. Transferring the incorrect embryo into a patient is an intolerable event for patients and the health care providers. It can expose reproductive endocrinologists and IVF facilities to substantial liability and financial burden. Obstetrics and Gynecology is traditionally one of the highest litigated medical specialties, and errors in embryo handling and lab practices were the most common reason for litigation against reproductive endocrinologists resulting in financial settlements.

It is estimated that nearly seventy-five percent of physicians had at least one professional liability claim against them in their career. Errors in the embryology laboratory were the most frequent cause for claims and accounted for thirty-eight percent of claims paid, with an overall incidence of 0.03% among the total IVF cycles. On average, each claim involving embryo issues resulted in a settlement of around two hundred thousand dollars. Such incidents and the subsequent public scrutiny may tarnish the reputation of even the most reputable IVF centers. Double-checking by a second operator, referred to as witnessing, is the main control measure used to avoid biological sample misidentification, but this is time-consuming, error prone, and a source of additional stress for embryologists. Similar problems exist for tracking other cell lines, for example, stem cell lines and cells used for genetic therapy.

Electronic witnessing systems (EWS) have been introduced as an alternative to mitigate misclassifications and mix-ups. EWS are based on radio frequency identification (RFID) tags to track and record patients and samples during IVF. Using RFID tags, a patient's identity is monitored at every stage of the treatment, and at the same time, the system captures information regarding the cycle progress and operator actions. The IVF witness system monitors every instance when gametes or embryos are transferred from one container to the next. These systems have also demonstrated to improve the laboratory efficiency by helping embryologists monitor the sequence, timing, and completion of procedures and by minimizing interruptions. However, all current systems are limited to tracking only the dish that contains the cells, not the cell itself, and thus can only aid in preventing patient mix-ups. Mix-ups at the cellular level are just as likely as patient mix-ups since embryologists have to manually track and consider the genetic profiles each individual embryo when making critical clinical decisions.

### SUMMARY

In accordance with one example, a digital witnessing system includes a processor and a non-transitory computer readable medium that stores instructions executable by the processor to provide an image interface configured to receive a first image and a similarity network. The similarity network compares the first image to a second image to determine a probability that the first image and the second image represent a same cellular structure or line of cellular structures. The similarity network is trained on a training set of samples, with each sample including two images and a binary label. The training set includes both positive examples, using two images of a same cellular structure or line of cellular structures, and negative examples, which use two images that are not of the same structure or the same line of cellular structures. An output device receives either the probability or a categorical parameter representing the categorical parameter from the similarity network.

In accordance with another example, a method includes receiving a first image of an embryo at a similarity network and comparing the first image to a second image at the similarity network to determine a probability that the second image represents the embryo. The similarity network is trained on a training set of samples, each sample including two images and a binary label. The training set includes both positive examples, using two images of a same embryo, and negative examples, which use two images that are of different embryos. The probability that the second image represents the embryo or a categorical parameter representing the probability is provided to an output device.

In accordance with a further example, a digital witnessing system includes an imager configured to generate a first image of an embryo, a processor, and a non-transitory computer readable medium storing instructions executable by the processor. The executable instructions include an image interface configured to receive the first image and a Siamese network that compares the first image to a second image to determine a probability that the second image represents the embryo. The Siamese network is trained using a cross-entropy loss function on a training set of samples, with each sample including two images and a binary label.

The training set including both positive examples, using two images of a same embryo, and negative examples, which use two images that are of different embryos. An output device that receives the probability or a categorical parameter representing the probability from the Siamese network.

### BRIEF DESCRIPTION OF THE DRAWINGS

FIG. 1 illustrates one example of a system for determining if two images represent a same cellular structure or line of cellular structures;

FIG. 2 illustrates an example of a system for determining if two images represent a same embryo;

FIG. 3 illustrates an example of a method for determining if two images represent a same cellular structure or line of cellular structures;

FIG. 4 illustrates an example of a method for determining if two images represent a same embryo; and

FIG. 5 is a schematic block diagram illustrating an exemplary system of hardware components capable of implementing examples of the systems and methods disclosed herein.

### DEFINITIONS

A “cellular structure,” as used herein, is a single cell or multi-cellular structure that contains less than five hundred cells. Any of a sperm cell, an oocyte, and an embryo at any stage of development are examples of a cellular structure.
A “similarity network” is an artificial neural network or set of integrated artificial neural networks that compares two images to determine a similarity metric between the two images.

### DETAILED DESCRIPTION

Systems and methods are provided herein for combining electronic witnessing through radio-frequency identification and visual embryo tracking through deep learning. Cell-level errors, similar to patient-level errors, have dire consequences to patients and the eventual newborn, but no current system offers cell-level witnessing, and the human eye cannot reliably differentiate embryos based on the visual presentation. Digital witnessing using morphological features is complicated due to these embryos both being similar to one another in appearance and the significant morphological changes that occur over the course of an IVF cycle. Therefore, traditional deep learning strategies and library-based identification systems do not work reliably.

The approaches used herein retain key features unique to each embryo while adapting to their constantly changing morphological features. Similarity networks are leveraged for development of a novel network for embryo identification. One example of similarity network is a Siamese network. Siamese networks accept distinct inputs but possess a common energy function at the conjoined layer. Owing to their unique design, Siamese networks, unlike traditional networks, can be trained on a similarity function which allows them to learn if two images are similar and associated. Furthermore, due its symmetricity, when evaluating two distinct images, the conjoining layer computes the same metric as if we were to present the same two images to the opposite network. Therefore, this strategy allows for visual comparison of different embryos and establishment of their unique identities based on their morphology.

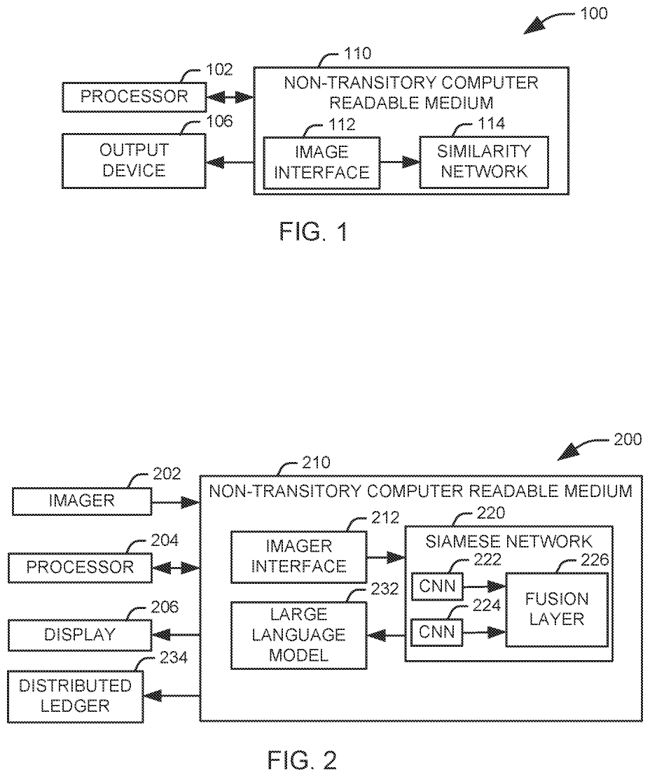

FIG. 1 illustrates one example of a system 100 for determining if two images represent a same cellular structure or line of cellular structures. The system 100 includes a processor 102, an output device 104, and a non-transitory computer readable medium 110 storing executable instructions, executed by the processor 102. It will be appreciated that the executable instructions can be spread across multiple non-transitory computer readable media that are operatively connected via an appropriate data connection, such that the executable instructions can be executed by multiple processors.

The executable instructions stored on the non-transitory computer readable medium 110 include an image interface 112 configured to receive at least one image. For example, the image interface 112 can receive the image or images from an imaging system or storage medium via a bus or network connection and condition the image for analysis at a similarity network 114. In one implementation, the image interface 112 can access a video stream of the cell in real-time and extract images or series of images from the video stream for analysis. The similarity network 114 compares two images, one or both of which are received at the image interface 112, to determine if they represent a same cellular structure or line of cellular structures. In one example, the similarity network 114 can also estimate a degree of development of the cellular structure that has taken place between capturing an earlier of the two images to the later of the two images. For example, a number of hours of development of an embryo can be estimated.

It will be appreciated that the similarly network can compare all or a portion of the provided images. In one example, the images can be segmented to isolate portions of the image that have been determined to be of particular significance in the similarity matching or to exclude portions of the image that may provide spurious results. For example, if two images of embryos are being compared, locations associated with procedures that the cell is undergoing or has recently completed (e.g., ICSI, freezing/thawing, laser hatching, biopsy) can be excluded to avoid inaccurate results due to morphological changes induced by these procedures.

Further, it will be appreciated that additional parameters can be provided to the similarity model to supplement the provided images. For example, the similarity network 114 can be provided with additional images using different modalities, timestamps for one or both cells, information about current or recent procedures performed on the cell, or any other relevant information for the similarity determination. Further, where the images are acquired from different sources, a domain transfer process, trained on corresponding images from the two source, can be used to transform one image as to emulate an image source associate with the other image.

In one example, the similarity network 114 is implemented as a convolutional neural network that includes two subnetworks that have identical layers and layer weights, referred to as a Siamese network. It will be appreciated, however, that the similarity network 114 can be implemented as any of a number of machine learning models, including triplet-based models, matching models, prototypical networks, contrastive learning, dual path models, deep metric learning models, vision transformers, and other comparison models. Further, the similarity network 114 can be trained on time series of images and use one or more models that can process sequences of images, including recurrent neural networks, three-dimensional convolutional neural networks, or three-dimensional vision transformers, or a combination of a spatial network, such as a convolutional neural network, with a temporal network, such as a recurrent neural network or a long short-term memory model, with the spatial model providing a representation of each image in the series to the temporal model for analysis. It will be appreciated that any appropriate loss function can be used in training the similarity network and that in one implementation, a binary cross-entropy loss function is utilized.

The output of the similarity network 114 can be provided to a user at the output device 104. In one example, the determined probability, or a categorical value representing the determined probability, can be provided to a user at a display. In one example, the categorical parameter can take on three values, one indicating that the two images represent the same cellular structure or line of cellular structures, a second value that indicates that two images do not represent the same cellular structure or line of cellular structures, and a third value indicating that the result is indeterminate. In one implementation, these values are determined by determining into which of three defined ranges that the determined probability falls.

In another example, the output device 104 can include a computer system operating a large language model that provides the probability or categorical parameter as part of a natural language report. This report can include not only the probability or representative parameter, but other parameters associated with the culturing and growth of cellular structure and one or more patients associated with the cellular structure. In one implementation, the large language model can be implemented as a multimodal large language model that also analyzes the image or other media associated with the image, such as associated text, images from other imaging systems (infra-red, fluorescence, etc.), or audio, to generate text representing other properties of the cellular structure, such as a quality of the cellular structure or a type or stage of development of the cellular structure. In a further example, the output device 104 can include a distributed ledger system that records the probability or representative parameter as an entry in the distributed ledger along with an identifier for a patient associated with the cellular structure.

In one implementation, the similarity network 114 can be one machine learning model of a plurality of neural networks or other machine learning models, each performing a separate task. For example, one model, for example, can receive an image of the cellular structure and produce an output representing a quality of the cellular structure. For example, the model can be implemented as a convolutional neural network that is trained on images of cellular structures with labels representing a quality of cellular structure provided by human experts. Another model can receive an image of the cellular structure and produce an output representing a developmental stage or age of the cellular structure. For example, the model can be implemented as a convolutional neural network that is trained on images of the cellular structures with labels representing the developmental stage or age of the cellular structure provided by human experts. A further model can receive an image of the cellular structure and produce an output representing genetic information associated with of the cellular structure. For example, the model can be implemented as a convolutional neural network that is trained on images of the cellular structures with labels representing one or more parameters associated with the genetics of the cellular structure.

In another implementation, the similarity network 114 can perform multiple functions along with identification or witnessing of the cellular structure. For example, the similarity network 114 can determine any or all of a developmental stage or age of a cellular structure, a quality of the cellular structure, and genetic information about the cellular structure in addition to determining if two images believed to be of the cellular structure actually represent the cellular structure. In this integrated similarity network 114, additional inputs beyond the images can be provided to the model including free text, images from other imaging systems (infra-red, fluorescence, etc.), audio, and numerical or categorical parameters representing any of the cellular structure, procedures performed on the cellular structure, a donor of the cellular structure or one of its components, or a recipient of the cellular structure. This contextual data can be provided manually by a user, pulled automatically from an electronic health records system, or provided by other feeder models.

It will be appreciated that the inclusion of additional tasks can provide additional context to the digital witnessing process. For example, the similarity network 114 can account for the age of a developing cellular structure and capture inconsistences between an expected age of the cellular structure, for example, from a time stamp associated with the image, and an age determined at the model. This particular capability can be included implicitly in the design of the similarity network 114. For example, three-dimensional vision transformers or convolutional neural networks can process series of images in a manner that hard codes the time-variable. Alternatively, the similarity network 114 can be trained to perform multiple tasks, with the loss function used to train the model including multiple terms focusing on specific tasks, including determining the developmental stage as well. This can also be employed to eliminate results that are inconsistent with morphokinetics and the developmental trajectory of the cellular structures. For example, a day five blastocyst should not regress to a cleavage-stage embryo, and an arrested two-cell embryo on day three will not progress to the blastocyst stage, and the similarity network 114 can be supplemented with a rule-based expert system to eliminate these inconsistent results. Similarly, the similarity network 114 can identify genetic abnormalities or other properties of the cellular structure from the images of the cellular structures and flag these for the attention of a user if they are inconsistent with expected values for the cellular structure. As a result, samples that haven't been ordered by a provider or that may pose a risk due to abnormal genetic results can be identified.

FIG. 2 illustrates an example of a system 200 for determining if two images represent a same embryo. It will be appreciated, for example, that the two images can represent two images of an embryo taken at different times during development or two images taken using different imagers or in different lighting conditions. In one implementation, the system 200 can be used to match an embryo that is at a development stage suitable for implantation (e.g., a blastocyst on day five) to an image from earlier in the development embryo. The system 200 includes an imager 202, a processor 204, a display 206, and a non-transitory computer readable medium 210 storing executable instructions, executed by the processor 204. It will be appreciated that the executable instructions can be spread across multiple non-transitory computer readable media that are operatively connected via an appropriate data connection, such that the executable instructions can be executed by multiple processors.

The imager 202 can include one or more cameras, capable of producing images in the visible or infrared range, paired with appropriate optics to provide an image of an embryo. In one example, the imager 202 records image data using a 20× objective lens at ten-minute intervals under illumination from a single 635 nm light emitting diode. Each patient's set of embryos are exported as videos using the imaging system software. In another implementation, intended for smaller clinics or other locations without a dedicated imager for embryos, the imager 202 can be implemented an attachment for a mobile device that operates with a camera of the mobile device to provide the embryo images. The housing for the attachment can be 3-D printed using polylactic acid. An acrylic lens can be included in the housing to provided appropriate magnification for the embryo images. Alternatively, the imager 202 can be implemented as a stand-alone system with an optical housing that contains an electronic circuit with a white light-emitting diode, a three-volt battery, and a single pole double-throw switch. The embryo sample is transilluminated, with a 10× Plan-Achromatic objective lens for image magnification and a complementary metal-oxide-semiconductor (CMOS) image sensor for embryo image data acquisition. The CMOS sensor can be connected to a single-board computer to process the captured images. The imager 202 can be connected to a mobile device or other computer system via a wireless connection (e.g., Wi-Fi, Bluetooth, or a similar connection) for data processing and visualization.

The executable instructions stored on the non-transitory computer readable medium 210 include an image interface 212 that receives the image of the embryo from the imager. It will be appreciated that the image interface 212 can receive the message directly from the imager 202, for example, via a bus or network connection, or indirectly via a non-transitory storage medium. The image interface 212 can also retrieve the one or more images from a local or remote storage medium, for example, a storage medium associated with an electronic health records system. It will be appreciated that the imager interface 212 can provide conditioning or formatting, such as cropping and filtering, for the received image for further processing. Further, where the image data is provided as a video, the image interface breaks the video of each individual embryo into frames to extract images from all timepoints post insemination, with the images are identified by their timestamps.

The image is provided to a Siamese network 220 that compares the first image to a second image to determine a probability that the second image represents the embryo. The second image, for example, can be retrieved by the image interface from memory and selected according to an identity of a patient associated with the embryo. The Siamese network 220 includes a first convolutional neural network 222 that receives the first image and generates a first feature embedding representing the first image and a second convolutional neural network 224 that receives the second image and generates a second feature embedding representing the second image. Each of the two convolutional neural networks 222 and 224 have identical layers and layer weights. A fusion layer 226 that generates the probability that the second image represents the same embryo from the first feature embedding and the second feature embedding. Each convolutional neural network 222 and 224 is a feed-forward artificial neural network that includes convolutional layers, which effectively apply a convolution to the values at the preceding layer of the network to emphasize various sets of features within an image. In a convolutional layer, each neuron is connected only to a proper subset of the neurons in the preceding layer, referred to as the receptive field of the neuron. An activation function, such as a rectified linear unit function, a sigmoid function, a hyperbolic tangent function, or a softmax function, can be applied after a set of one or more convolutional layers. At least one chromatic value (e.g., a value for an RGB color channel, a YCrCb color channel, or a grayscale brightness) associated with each pixel of the input image is provided as an initial input to the convolutional neural network.

The Siamese network 220 is trained using a cross-entropy loss function on a training set of samples, with each sample including two images and a binary label. The training set includes both positive examples, using two images of a same embryo, and negative examples, which use two images that are of different embryos. In one example, around 3.5 million images of around five thousand unique embryos were used for training. Training will be performed for around five hundred epochs, and with a validation error monitored on sets of randomly generated embryo image pairs in the validation set. Network training was stopped when error saturation was achieved. In some implementations, some layers of the convolutional neural networks 222 and 224 can be trained via transfer learning from another system, with only some of the layers trained on the training images.

The output of the Siamese network 220 is provided to a user at the display 204. The output is also provided to a large language model 232. The large language model 232 generates a natural language report from the determined probability, the first and/or second image, and data retrieved from an electronic health records system (not shown). It will be appreciated that the large language model 232 can be trained on reports generated by embryologists for previous patents. This can include associated with the donor of the gametes used to create the embryo, data associated with the woman into whom the embryo will be implanted, and data associated with the fertilization of the oocyte and its development. In one example, the natural language report includes the probability, a temperature at which the embryo is maintained, information about a chemical composition of the growth medium, and the name of a physician performing the fertilization. The output of the Siamese network 220 can also be provided to a distributed ledger 234 to maintain a transparent and inviolate record of the identity of the embryo. An entry on the distributed ledger 234 can include, for example, the probability generated at the Siamese network 220 as well as an identify of a woman into whom the embryo is to be implanted.

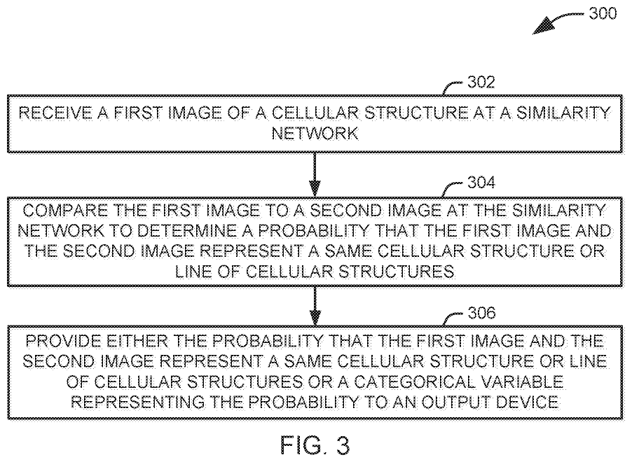

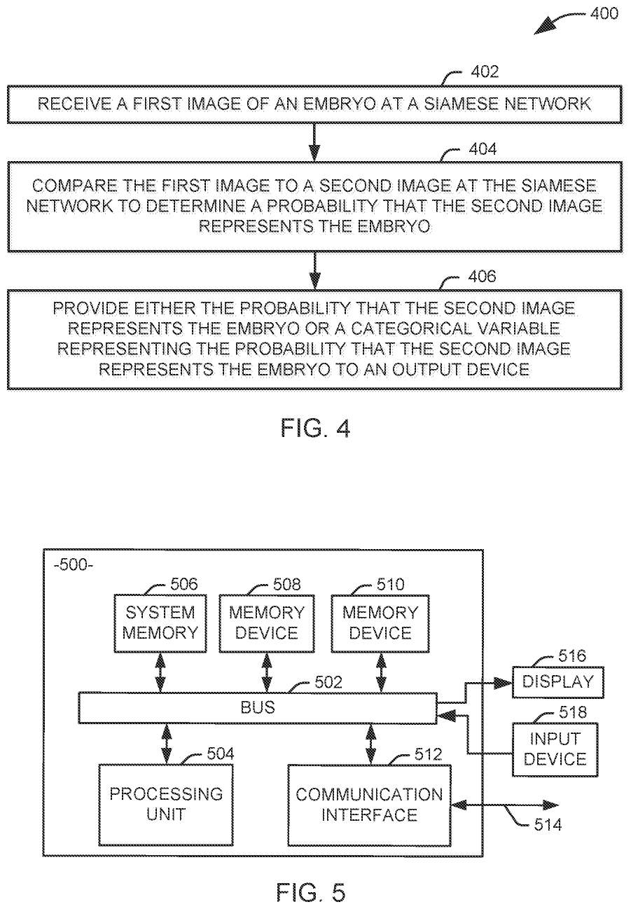

In view of the foregoing structural and functional features described above, methods in accordance with various aspects of the present invention will be better appreciated with reference to FIGS. 3 and 4. While, for purposes of simplicity of explanation, the methods of FIGS. 3 and 4 are shown and described as executing serially, it is to be understood and appreciated that the present invention is not limited by the illustrated order, as some aspects could, in accordance with the present invention, occur in different orders and/or concurrently with other aspects from that shown and described herein. Moreover, not all illustrated features may be required to implement a method in accordance with an aspect of the present invention.

FIG. 3 illustrates an example of a method 300 for determining if two images represent a same cellular structure or line of cellular structures. At 302, a first image of a cellular structure is received at a similarity network. In one implementation, the first image is received from an associated imager. In another implementation, the first image is retrieved from an associated computer memory. It will be appreciated that the first image can be extracted in real-time from a video of the cellular structure as an image frame or series of image frames. At 304, the first image is compared to a second image to determine a probability that the first image and the second image represent a same cellular structure or line of cellular structures. In one example, the first image can be segmented to provide an image segment that is compared to a corresponding segment of the second image. The similarity network is trained on a training set of samples, each sample including two images and a binary label, with the training set including both positive examples, using two images of a same cellular structure or line of cellular structures, and negative examples, which use two images that are not the same structure or from the same line of cellular structures. In one example, a binary cross entropy loss function is used for training. In one example, the similarity network includes a first convolutional neural network that receives the first image and generates a first feature embedding, a second convolutional neural network that receives the second image and generates a second feature embedding, and a fusion layer that generates the probability that the first image and the second image represent the same cellular structure or line of cellular structures from the first feature embedding and the second feature embedding.

At 306, either the probability that the first image and the second image represent a same cellular structure or line of cellular structures or a categorical variable representing the probability are provided to an output device from the similarity network. In one implementation, the similarity network can also estimate a degree of development of the cellular structure that has taken place between capturing an earlier of the two images to the later of the two images. For example, a number of hours of development of an embryo can be estimated. In one implementation, when it is determined that the first image and the second image do not represent the same cellular structure or line of cellular structures, the first image can be compared to different samples from a library of cellular structures to attempt to identify the first image. In one example, the samples can be selected for comparison based upon a similarity with the expected identity of the sample. In this example, an image believed to be of a blastocyst can be compared with other images of blastocysts to more efficiently identify the sample. In another example, the samples can be selected for comparison based upon a parameter generated by the similarity network, for example, related to the stage of development or genetic defect associated with the cellular structure depicted in the first image.

In one example, the output device is a display that provides the probability or the categorical variable representing the probability to a user. Additional or alternatively, the output device can be a distributed ledger, with the probability or the categorical variable representing the probability provided as an entry on the distributed ledger along with a value representing a patient associated with the cellular structure. Similarly, the output device can be a multi-modal large language model that provides a natural language report to a user describing at least the one of the probability and the categorical variable representing the probability. In practice, the natural language report can contain other information as well, for example, as received from sensors associated with a growth medium and incubation environment associated with the cellular structure, other imaging of the cellular structures, and information retrieved from an electronic health records system.

FIG. 4 illustrates an example of a method 400 for determining if two images represent a same embryo. At 402, a first image of an embryo is received at a Siamese network. In one example, the image is received from an associated imager. At 404, the first image is compared to a second image at the Siamese network to determine a probability that the second image represents the embryo. In one example, the second image can be an image of an embryo taken at a known stage of development, and the first image, taken at a later stage of development, can be compared to the second image to determine if they represent the same embryo at different stages of development. The Siamese network is trained on a training set of samples, with each sample including two images and a binary label. The training set includes both positive examples, using two images of a same embryo, and negative examples, which use two images that are of different embryos. In one example, the Siamese network is trained using a binary cross entropy loss function.

At 406, the probability that the second image represents the embryo is provided to an output device. In one example, the output of the Siamese network is provided to a user at the display. Additionally or alternatively, the output of the Siamese network can be provided to a large language model that generates a natural language report from the determined probability, the first and/or second image, and data retrieved from an electronic health records system (not shown), and data received from one or more sensors associated with the incubation environment for the embryo. For example, the report can include a temperature of the growth medium, a chemical composition of the growth medium, and an identity of a physician who performed the fertilization procedure to create the embryo along with the generated probability. In still another alternative or addition, the output of the Siamese network can be provided to a distributed ledger to maintain a transparent and inviolate record of the identity of the embryo. An entry on the distributed ledger can include, for example, the probability generated at the Siamese network as well as an identify of a woman into whom the embryo is to be implanted.

FIG. 5 is a schematic block diagram illustrating an exemplary system 500 of hardware components capable of implementing examples of the systems and methods disclosed in FIGS. 1-4, such as the cellular digital fingerprinting system illustrated in FIG. 1. The system 500 can include various systems and subsystems. The system 500 can be any of personal computer, a laptop computer, a workstation, a computer system, an appliance, an application-specific integrated circuit (ASIC), a server, a server blade center, or a server farm.

The system 500 can includes a system bus 502, a processing unit 504, a system memory 506, memory devices 508 and 510, a communication interface 512 (e.g., a network interface), a communication link 514, a display 516 (e.g., a video screen), and an input device 518 (e.g., a keyboard and/or a mouse). The system bus 502 can be in communication with the processing unit 504 and the system memory 506. The additional memory devices 508 and 510, such as a hard disk drive, server, stand-alone database, or other non-volatile memory, can also be in communication with the system bus 502. The system bus 502 interconnects the processing unit 504, the memory devices 506-510, the communication interface 512, the display 516, and the input device 518. In some examples, the system bus 502 also interconnects an additional port (not shown), such as a universal serial bus (USB) port.

The system 500 could be implemented in a computing cloud. In such a situation, features of the system 500, such as the processing unit 504, the communication interface 512, and the memory devices 508 and 510 could be representative of a single instance of hardware or multiple instances of hardware with applications executing across the multiple of instances (i.e., distributed) of hardware (e.g., computers, routers, memory, processors, or a combination thereof). Alternatively, the system 500 could be implemented on a single dedicated server.

The processing unit 504 can be a computing device and can include an application-specific integrated circuit (ASIC). The processing unit 504 executes a set of instructions to implement the operations of examples disclosed herein. The processing unit can include a processing core.

The additional memory devices 506, 508, and 510 can store data, programs, instructions, database queries in text or compiled form, and any other information that can be needed to operate a computer. The memories 506, 508 and 510 can be implemented as computer-readable media (integrated or removable) such as a memory card, disk drive, compact disk (CD), or server accessible over a network. In certain examples, the memories 506, 508 and 510 can comprise text, images, video, and/or audio, portions of which can be available in formats comprehensible to human beings. Additionally or alternatively, the system 500 can access an external data source or query source through the communication interface 512, which can communicate with the system bus 502 and the communication link 514.

In operation, the system 500 can be used to implement one or more parts of a digital fingerprinting system in accordance with the present invention. Computer executable logic for implementing the composite applications testing system resides on one or more of the system memory 506, and the memory devices 508, 510 in accordance with certain examples. The processing unit 504 executes one or more computer executable instructions originating from the system memory 506 and the memory devices 508 and 510. It will be appreciated that a computer readable medium can include multiple computer readable media each operatively connected to the processing unit.

Implementation of the techniques, blocks, steps, and means described above can be done in various ways. For example, these techniques, blocks, steps, and means can be implemented in hardware, software, or a combination thereof.

For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes, but is not limited to portable or fixed storage devices, optical storage devices, wireless channels, and/or various other storage mediums capable of storing that contain or carry instruction(s) and/or data.

In the preceding description, specific details have been set forth in order to provide a thorough understanding of example implementations of the invention described in the disclosure. However, it will be apparent that various implementations may be practiced without these specific details. For example, circuits, systems, networks, processes, and other components may be shown as components in block diagram form in order not to obscure the example implementations in unnecessary detail. In other instances, well-known circuits, processes, algorithms, structures, and techniques may be shown without unnecessary detail in order to avoid obscuring the examples. The description of the example implementations will provide those skilled in the art with an enabling description for implementing an example of the invention, but it should be understood that various changes may be made in the function and arrangement of elements without departing from the spirit and scope of the invention. Accordingly, the present invention is intended to embrace all such alterations, modifications, and variations that fall within the scope of the appended claims.

What is claimed is:

1. A digital witnessing system comprising:
a processor;
a non-transitory computer readable medium storing instructions executable by the processor, the executable instructions comprising:
an image interface configured to receive a first image; and
a similarity network that compares the first image to a second image to determine a probability that the first image and the second image represent a same cellular structure or line of cellular structures, the similarity network being trained on a training set of samples, each sample including two images and a binary label, the training set including both positive examples, using two images of a same cellular structure or line of cellular structures, and negative examples, which use two images that are not of the same structure or line of cellular structures; and
an output device that receives one of the probability and a categorical variable representing the probability from the similarity network.

2. The digital witnessing system of claim 1, wherein the similarity network is trained using a binary cross entropy loss function.

3. The digital witnessing system of claim 1, wherein the first image is one image of a first series of images and the second image is one image of a second series of images, the similarity network being implemented as one of a three-dimensional vision transformer, a three-dimensional convolutional neural network, and a recurrent neural network and comparing the first series of images to the second series of images to determine a probability that the first series of images and the second series of images represent the same cellular structure or line of cellular structures.

4. The digital witnessing system of claim 1, wherein the image interface is configured to retrieve the first image as at least one frame from a video of the cellular structure.

5. The digital witnessing system of claim 1, wherein the output device is a display that provides the one of the probability and the categorical variable representing the probability to a user.

6. The digital witnessing system of claim 1, wherein the first image is an image of an embryo, and the similarity network determines a probability that the second image is an image of the embryo.

7. The digital witnessing system of claim 1, wherein the output device is a distributed ledger, the one of the probability and the categorical variable representing the probability being provided as an entry on the distributed ledger along with a value representing a patient associated with the cellular structure.

8. The digital witnessing system of claim 1, wherein the output device is a large language model that provides a natural language report to a user describing at least the one of the probability and the categorical variable representing the probability.

9. The digital witnessing system of claim 1, wherein the similarity network comprises a Siamese network, the Siamese network comprising:
a first convolutional neural network that receives the first image and generates a first feature embedding;
a second convolutional neural network that receives the second image and generates a second feature embedding; and
a fusion layer that generates the probability that the first image and the second image represent the same cellular structure or line of cellular structures from the first feature embedding and the second feature embedding.

10. A method comprising:
receiving a first image of an embryo at a similarity network;
comparing the first image to a second image at the similarity network to determine a probability that the second image represents the embryo, wherein the similarity network is trained on a training set of samples, each sample including two images and a binary label, the training set including both positive examples, using two images of a same embryo, and negative examples, which use two images that are of different embryos; and
providing one of the probability that the second image represents the embryo and a categorical variable representing the probability that the second image represents the embryo to an output device.

11. The method of claim 10, wherein the similarity network is trained using a binary cross entropy loss function.

12. The method of claim 10, wherein comparing the first image to the second image at the similarity network to determine the probability that the second image represents the embryo further comprising the first image to the second image at the similarity network to determine a degree of development of the embryo between the first image and the second image.

13. The method of claim 10, wherein providing the one of the probability that the second image represents the embryo and the categorical variable representing the probability that the second image represents the embryo comprises generating an entry on a distributed ledger comprising the one of the probability that the second image represents the embryo and the categorical variable representing the probability that the second image represents the embryo and a value representing a patient associated with the embryo.

14. The method of claim 10, wherein providing the probability that the second image represents the embryo to the output device comprises generating a natural language report at a large language model that includes the one of the probability that the second image represents the embryo and the categorical variable representing the probability that the second image represents the embryo and one or more additional parameters representing the embryo.

15. The method of claim 10, further comprising segmenting the first image to provide an image segment, wherein comparing the first image to the second image at the similarity network comprises comparing the image segment to a corresponding segment of the second image.

16. A digital witnessing system comprising:
an imager configured to generate a first image of an embryo;
a processor;
a non-transitory computer readable medium storing instructions executable by the processor, the executable instructions comprising:
an image interface configured to receive the first image; and
a Siamese network that compares the first image to a second image to determine a probability that the second image represents the embryo, the Siamese network being trained using a cross-entropy loss function on a training set of samples, each sample including two images and a binary label, the training set including both positive examples, using two images of a same embryo, and negative examples, which use two images that are of different embryos; and
an output device that receives the one of the probability that the second image represents the embryo and a categorical variable representing the probability that the second image represents the embryo from the Siamese network.

17. The digital witnessing system of claim 16, the Siamese network comprising:
a first convolutional neural network that receives the first image and generates a first feature embedding;
a second convolutional neural network that receives the second image and generates a second feature embedding; and
a fusion layer that generates the probability that the second image represents the same embryo from the first feature embedding and the second feature embedding.

18. The digital witnessing system of claim 16, wherein the image interface is configured to retrieve the second image from an associated computer memory.

19. The digital witnessing system of claim 16, wherein the output device is a distributed ledger, the one of the probability that the second image represents the embryo and the categorical variable representing the probability that the second image represents the embryo being provided as an entry on the distributed ledger along with a value representing a patient associated with the embryo.

20. The digital witnessing system of claim 16, wherein the output device is a large language model that provides a natural language report to a user describing at least the one of the probability that the second image represents the embryo and the categorical variable representing the probability that the second image represents the embryo and one or more additional parameters representing the embryo.
